(19) United States
(12) Patent Application Publication
(10) Pub. No.: US 2025/0005746 A1
(43) Pub. Date: Jan. 2, 2025

(54) TYMPANUM IMAGE PROCESSING APPARATUS AND METHOD FOR GENERATING NORMAL TYMPANUM IMAGE BY USING MACHINE LEARNING MODEL TO OTITIS MEDIA TYMPANUM IMAGE

(71) Applicants: THE ASAN FOUNDATION, Seoul (KR); UNIVERSITY OF ULSAN FOUNDATION FOR INDUSTRY COOPERATION, Ulsan (KR)

(72) Inventors: Jihoon KWEON, Seoul (KR); Joong-Ho AHN, Seoul (KR); Yeonjoo CHOI, Seoul (KR); Keunwoo PARK, Seoul (KR); Jihye CHAE, Seoul (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 18/681,973

(22) PCT Filed: Dec. 6, 2021
(86) PCT No.: PCT/KR2021/018349

§ 371 Date: Feb. 7, 2024

(30) Foreign Application Priority Data
Aug. 26, 2021 (KR) 10-2021-0112900

(51) Int. Cl. G06T 7/00; G06T 3/14; G06T 11/60; G06V 10/422; G06V 10/70; G16H 70/20

(52) U.S. Cl. CPC G06T 7/0012; G06T 3/14; G06T 11/60; G06V 10/422; G06V 10/70; G16H 70/20; G06T 2207/20081; G06T 2207/30004; G06V 2201/03

(57) ABSTRACT

A tympanum image processing apparatus may comprise: a processor which extracts, from a tympanum image, a tympanum outline of the tympanum image and earwax region of the tympanum image by using a first machine learning model, obtains, on the basis of the tympanum outline of the tympanum image, a target image of the entire tympanum, a tympanum outline of the target image, and earwax region of the target image, and generates a transformed image in which an abnormal region of the target image is changed to a normal region; and a display which displays at least one of the transformed image and the target image so that a tympanum region of the transformed image is aligned at a position corresponding to the position of a tympanum region of the target image.

### TECHNICAL FIELD

Hereinafter, technology related to tympanum image processing is provided.

### BACKGROUND ART

Acute otitis media is a disease that occurs so commonly that 80% of children before the age of 3 experience otitis media, has frequent recurrences, and requires the use of many antibiotics. Otitis media with effusion is a disease that occurs when effusion accumulates in the tympanum due to sequelae of acute otitis media or a poor function of the middle ear ventilating tube. Otitis media with effusion is known to be the most common cause of hearing loss in children. Suppurative otitis media, which causes a perforation in the tympanum and inflammation of the tympanic cavity and hearing loss, and otitis media with cholesteatoma, which causes a portion of the tympanum to collapse and destroy the surrounding bone, causing hearing loss and facial paralysis in severe cases, happen not infrequently.

To diagnose otitis media in hospitals, an endoscopy that obtains an image close to the tympanum through the external auditory canal is generally used. It is used in a variety of hospitals, including pediatrics and family medicine, and is often equipped in private hospitals as well. Recently, endoscopy in the form of a portable device connected to a personal communication device has been developed, leading to increasing opportunities to obtain an image of the tympanum.

However, otitis media has many different types of disease, so there are many cases in which it is difficult for even experienced specialists to make an accurate diagnosis. Recently, with the development of deep learning technology, the technology for classifying major diseases has shown high performance, but it may not support diagnosis for diseases that are not considered the target of learning. Therefore, there is an urgent need to develop a method that may effectively provide information related to the occurrence of a disease or abnormality.

### DISCLOSURE OF THE INVENTION

### Technical Solutions

An apparatus for processing a tympanum image according to an embodiment includes a processor configured to extract, from a tympanum image, a tympanum outline of the tympanum image and an earwax region of the tympanum image using a first machine learning model, obtain a target image for an entire tympanum, a tympanum outline of the target image, and an earwax region of the target image based on the tympanum outline of the tympanum image, and generate a transformed image in which an abnormal region of the target image is changed to a normal region and includes a display configured to display at least one of the transformed image and the target image so that a tympanum region of the transformed image is aligned at a position corresponding to a position of a tympanum region of the target image.

The display may be configured to display a graphic object indicating the abnormal region on the target image and display a graphic object indicating a region in which the abnormal region is replaced by the normal region on the transformed image.

The processor may be configured to determine whether the tympanum image is about an entire tympanum based on the tympanum outline of the tympanum image and determine the target image based on the tympanum image in response to determining that the tympanum image is about an entire tympanum.

The processor may be configured to determine whether the tympanum image is about an entire tympanum based on the tympanum outline of the tympanum image, obtain an additional tympanum image in response to determining that the tympanum image is about a portion of a tympanum, extract a tympanum outline of the additional tympanum image and an earwax region of the additional tympanum image from the additional tympanum image using the first machine learning model, update a temporary image by stitching the additional tympanum image to the tympanum image, determine whether the temporary image is about an entire tympanum based on a tympanum outline of the temporary image, and determine the target image based on the temporary image in response to determining that the temporary image is about an entire tympanum.

The processor may be configured to generate the transformed image by inputting the target image to a second machine learning model in response to a case in which a ratio of a region occluded by earwax is less than a threshold ratio compared to a region of the entire tympanum in the target image.

The processor may be configured to calculate an objective function value between a temporary output image generated by applying the second machine learning model to a training abnormal tympanum image and a ground truth tympanum image and to repeatedly update a parameter of the second machine learning model so that the calculated objective function value converges.

The processor may be configured to generate the transformed image by inputting the target image, the tympanum outline of the target image, and the earwax region of the target image to a second machine learning model.

The processor may be configured to calculate an objective function value between a temporary output image generated by applying the second machine learning model to a training abnormal tympanum image and a ground truth tympanum image, a tympanum outline of the training abnormal tympanum image, and an earwax region of the training abnormal tympanum image and to repeatedly update a parameter of the second machine learning model so that the calculated objective function value converges.

The processor may be configured to repeatedly update a parameter of the first machine learning model so that an objective function value between temporary output data including a tympanum outline and an earwax region extracted using the first machine learning model from a training tympanum image and ground truth data converges.

The processor may be configured to provide an earwax removal guide in response to a case in which a ratio of a region occluded by earwax is greater than or equal to a threshold ratio compared to a region of the entire tympanum in the target image, and the display may be configured to display the target image and the earwax removal guide.

The processor may be configured to select one similar tympanum image from among a plurality of normal tympanum images based on at least one of age, gender, and race of a user in response to a case in which a ratio of a region occluded by earwax is greater than or equal to a threshold ratio compared to a region of the entire tympanum in the target image, and the display may be configured to display the similar tympanum image and the target image by aligning a tympanum region of the similar tympanum image at a position corresponding to the position of the tympanum region of the target image.

A method of processing a tympanum image according to an embodiment includes extracting, from a tympanum image, a tympanum outline of the tympanum image and an earwax region of the tympanum image using a first machine learning model, obtaining a target image of an entire tympanum, a tympanum outline of the target image, and an earwax region of the target image based on the tympanum outline of the tympanum image, generating a transformed image in which an abnormal region of the target image is changed to a normal region, and displaying at least one of the transformed image and the target image so that a tympanum region of the transformed image is aligned at a position corresponding to a position of a tympanum region of the target image.

### BRIEF DESCRIPTION OF DRAWINGS

FIG. 1 illustrates a tympanum image processing apparatus according to an embodiment.

FIG. 2 illustrates a flowchart showing a tympanum image processing method to generate a transformed image, which is an image about a virtual tympanum without abnormality.

FIG. 3 illustrates a tympanum image about the entire tympanum and a tympanum outline and an earwax region extracted from the tympanum image using a first machine learning model, according to an embodiment.

FIG. 4 illustrates a tympanum image about a portion of the tympanum and a tympanum outline and an earwax region extracted from the tympanum image using a first machine learning model, according to an embodiment.

FIG. 5 illustrates an operation in which the tympanum image processing apparatus determines a target image depending on whether a tympanum image is about the entire tympanum, according to an embodiment.

FIG. 6 illustrates a flowchart showing an operation of determining a target image based on a temporary image by generating the temporary image for the entire tympanum, according to an embodiment.

FIG. 7A illustrates an earwax region and a tympanum outline overlaid on a tympanum image for a portion of the tympanum, according to an embodiment.

FIG. 7B illustrates an earwax region and a tympanum outline overlaid on an additional tympanum image, according to an embodiment.

FIG. 7C illustrates an earwax region and a tympanum outline overlaid on a temporary image that is updated by stitching the additional tympanum image of FIG. 7B to the tympanum image of FIG. 7A.

FIG. 8 illustrates a target image, a tympanum outline of a target image, an earwax region of a target image, and a region where a tympanum region is occluded by an earwax region, according to an embodiment.

FIG. 9 illustrates a transformed image generated by inputting a target image about the entire tympanum to a second machine learning model and a tympanum outline of a transformed image, according to an embodiment.

FIG. 10 illustrates a display displaying a transformed image and a target image according to an embodiment.

FIG. 11 illustrates a display including a first region and a second region divided by a reference line, according to an embodiment.

FIG. 12 illustrates a display overlaying and displaying a graphic object indicating a certain region on at least one of a target image and a transformed image, according to an embodiment.

FIG. 13A illustrates a display displaying a target image in response to a target image display input, according to an embodiment.

FIG. 13B illustrates the display of FIG. 13A displaying a transformed image in response to a transformed image display input.

FIG. 14 illustrates a flowchart of a tympanum image processing method that displays a target image and a similar tympanum image in response to a case in which the ratio of a region occluded by earwax is greater than or equal to a threshold ratio compared to a region of the entire tympanum in the target image, according to an embodiment.

FIG. 15 illustrates a flowchart of a tympanum image processing method that displays a target image and a guide in response to a case in which the ratio of a region occluded by earwax is greater than or equal to a threshold ratio compared to a region of the entire tympanum in the target image, according to an embodiment.

### BEST MODE FOR CARRYING OUT THE INVENTION

The following structural or functional descriptions of embodiments described herein are merely intended for the purpose of describing the embodiments described herein and may be implemented in various forms. Accordingly, the embodiments are not construed as limited to the disclosure and should be understood to include all changes, equivalents, and replacements within the idea and the technical scope of the disclosure.

Although terms, such as first, second, and the like are used to describe various components, the components are not limited to the terms. These terms should be used only to distinguish one component from another component. For example, a first component may be referred to as a second component, or similarly, the second component may be referred to as the first component.

It should be noted that if it is described that one component is “connected”, “coupled”, or “joined” to another component, a third component may be “connected”, “coupled”, and “joined” between the first and second components, although the first component may be directly connected, coupled, or joined to the second component.

The singular forms “a”, “an”, and “the” are intended to include the plural forms as well, unless the context clearly indicates otherwise. It will be further understood that the terms “comprises/including” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms, including technical and scientific terms, used herein have the same meaning as commonly understood by one of ordinary skill in the art to which this disclosure pertains. Terms, such as those defined in commonly used dictionaries, should be construed to have meanings matching with contextual meanings in the relevant art, and are not to be construed to have an ideal or excessively formal meaning unless otherwise defined herein.

Hereinafter, the embodiments will be described in detail with reference to the accompanying drawings. When describing the embodiments with reference to the accompanying drawings, like reference numerals refer to like components and a repeated description related thereto will be omitted.

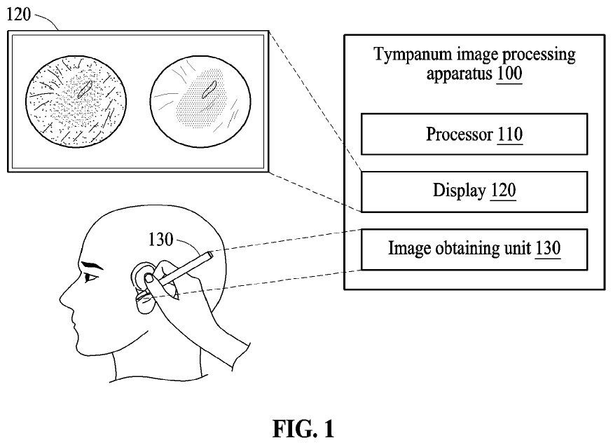

FIG. 1 illustrates a tympanum image processing apparatus according to an embodiment.

The tympanum image processing apparatus 100 according to an embodiment may include a processor 110, a display 120, and an image obtaining unit 130.

The image obtaining unit 130 may obtain a tympanum image of a target patient. The image obtaining unit 130 may generate a tympanum image by self-capturing the tympanum image or may receive a tympanum image from an external source.

For example, the image obtaining unit 130 may include a camera that captures an image and may be in a form capable of being inserted into the external auditory canal of the target patient. The image obtaining unit 130 may include a lighting unit that flashes light in a direction corresponding to the principal axis of the camera to assist capturing by the above-described camera. The image obtaining unit 130 may be inserted into the external auditory canal of the target patient by manipulation of a user. The image obtaining unit 130 including the camera and the lighting unit may capture a tympanum image in response to a capturing input of the user while being inserted into the external auditory canal of the target patient.

In another example, the image obtaining unit 130 may include a communication unit that receives a tympanum image captured by an external device from the external device (e.g., a separate device including a camera) that captures an image. The external device may be in a form capable of being inserted into the external auditory canal of the target patient. The communication unit may establish wired communication and/or wireless communication with an external device and receive a tympanum image from the external device. For example, a region (hereinafter, referred to as an ‘abnormal region’) in which at least one of diseases related to the tympanum of the target patient and state abnormalities appears may be displayed on the tympanum image.

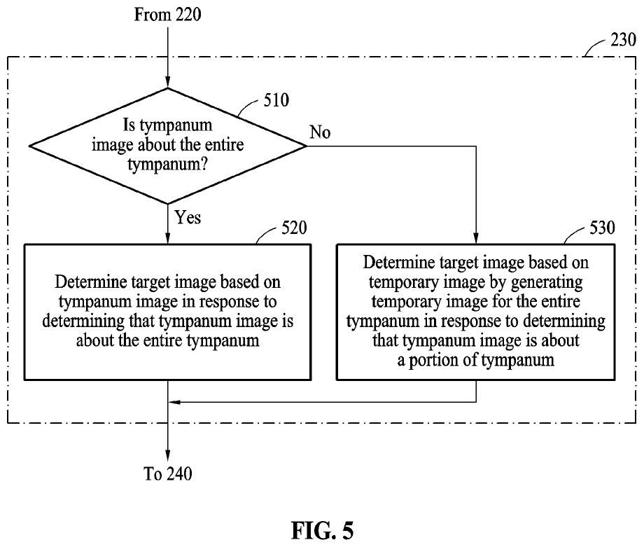

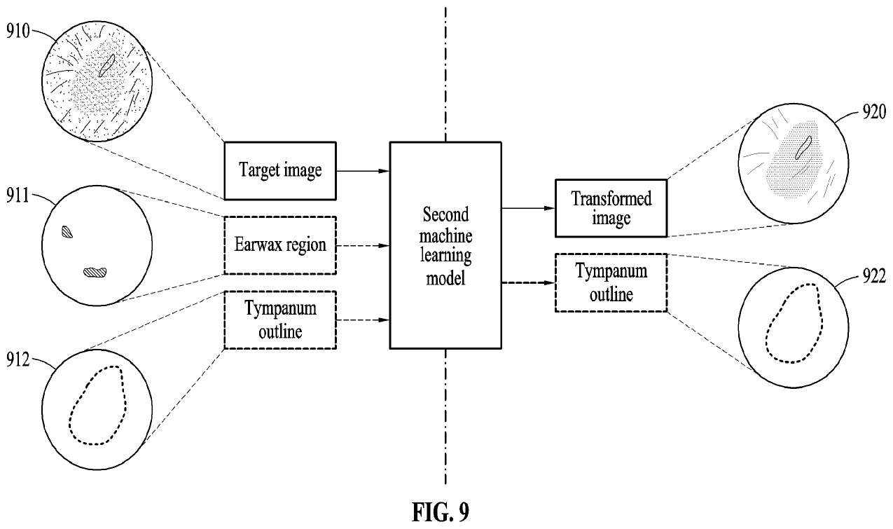

The processor 110 may generate a transformed image based on a target image. The obtaining of the target image is described below with reference to FIGS. 5 to 7, and the generating of the transformed image is described below with reference to FIG. 9.

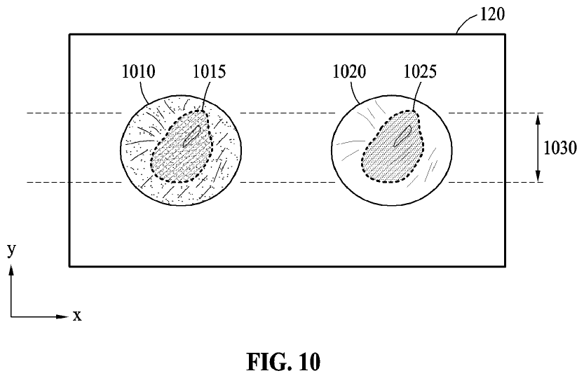

The display 120 may display at least one of the obtained target image and the transformed image. The display 120 according to an embodiment may display the transformed image at a position corresponding to the obtained target image. The displaying of at least one of the target image and the transformed image by the display 120 is described below with reference to FIGS. 10 to 13.

As described below; the tympanum image processing apparatus 100 may generate an image about a normal tympanum by changing an abnormal region to a normal region from a tympanum image including the abnormal region. The tympanum image processing apparatus 100 may display the obtained tympanum image and an image about the tympanum of which the abnormal region is changed. The tympanum image processing apparatus 100 according to an embodiment may provide the user with an intuitive and convenient comparison interface between a captured tympanum image and an image about a normal tympanum.

Operations of the tympanum image processing apparatus 100 are described in detail below.

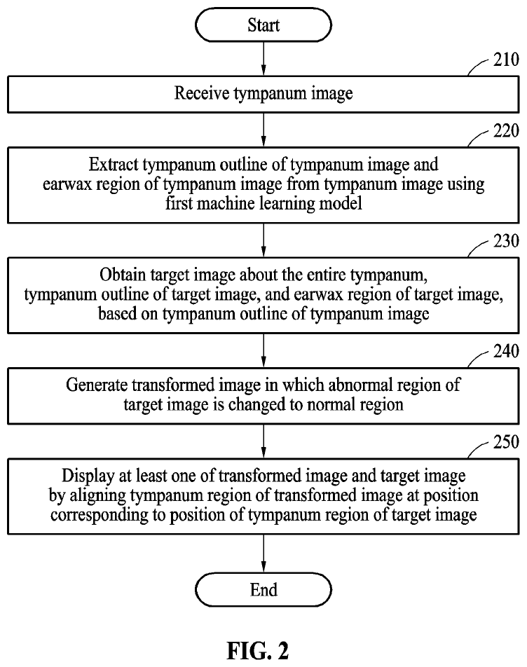

FIG. 2 illustrates a flowchart showing a tympanum image processing method to generate a transformed image, which is an image about a virtual tympanum without abnormality:

In operation 210, an image obtaining unit (e.g., the image obtaining unit 130 in FIG. 1) may obtain a tympanum image. For example, the tympanum image may include at least a portion of the entire tympanum. Due to a manipulation error of the user, an environmental factor such as insufficient light amount, and movement of a patient, only a portion of the entire tympanum may be captured.

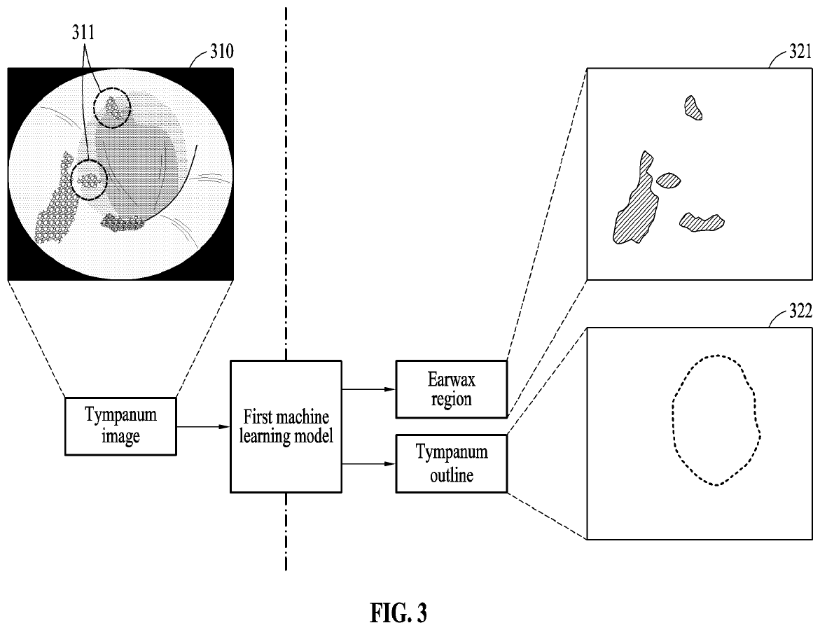

In operation 220, a processor (e.g., the processor 110 in FIG. 1) may extract a tympanum outline and an earwax region from a tympanum image using a first machine learning model. The tympanum outline may be a boundary line that divides a region about the tympanum from the other regions (e.g., the external auditory canal and regions of other parts) in the tympanum image. The tympanum outline may be extracted from the tympanum image. For example, the tympanum outline may be a set of pixels corresponding to the boundary portions of the tympanum in the tympanum image. The earwax region may be a region corresponding to earwax in the tympanum image. For example, the earwax region may be a set of pixels corresponding to earwax in the tympanum image. For reference, when a portion of the tympanum outline is occluded by earwax, the portion occluded by earwax at the tympanum outline may be estimated based on the first machine learning model. The first machine learning model and extraction of the tympanum outline and the earwax region are described below with reference to FIG. 3.

In operation 230, the processor may obtain a target image about the entire tympanum based on the tympanum outline of the tympanum image. The target image may be an image including a region of a target part provided to the user (e.g., the user of the tympanum image processing apparatus) among body parts of a subject. The subject may be a person who is a target of the tympanum image capturing that is input to the tympanum image processing apparatus. For example, the user may be a guardian, the subject may be an infant to be captured, and the target part may be a part including the tympanum of the infant. An image in which at least a portion of the tympanum is captured may be referred to as a tympanum image. The tympanum image processing apparatus may provide the guardian with information for the intuitive comparison between the tympanum image of the infant and a transformed image. However, embodiments are not limited thereto, and the subject may be the user also. The obtaining of the target image is described below with reference to FIG. 5.

In operation 240, the processor may generate a transformed image in which an abnormal region of the target image is changed to a normal region. The transformed image may be an image in which the earwax region of the target image is changed to a normal region. The generating of the transformed image is described below with reference to FIG. 9.

In operation 250, a display may display at least one of the transformed image and the target image so that a tympanum region of the transformed image is aligned at a position corresponding to a position of a tympanum region of the target image. The displaying of at least one of the transformed image and the target image is described below with reference to FIGS. 10 to 13.

FIG. 3 illustrates a tympanum image 310 about the entire tympanum and a tympanum outline 322 and an earwax region 321 extracted from the tympanum image 310 using the first machine learning model, according to an embodiment.

As described above in operation 220 of FIG. 2, a processor according to an embodiment may extract the tympanum outline and the earwax region of the tympanum image. For example, the processor may calculate the tympanum outline and the earwax region from the tympanum image based on the first machine learning model.

In FIG. 3, an example in which the tympanum and/or a portion of the tympanum outline are occluded by earwax in the tympanum image is described, as shown in a region 311. The processor may estimate a portion of the tympanum outline corresponding to the region 311 using the first machine learning model. The processor may extract the entire tympanum outline by estimating a portion of the tympanum outline even when a portion of a region corresponding to the boundary of the tympanum is occluded by earwax in the tympanum image.

The first machine learning model is a model designed and trained to extract the tympanum outline and the earwax region from the tympanum image and, for example, may include a neural network. The schematic structure of the neural network is described as follows. According to an embodiment, the neural network may include a plurality of layers including a plurality of nodes. Additionally, the neural network may include connection weights that connect the plurality of nodes included in each of the plurality of layers to nodes included in another layer. For example, the neural network may refer to a recognition model that mimics the computational capability of a biological system using a large number of nodes connected via edges. The neural network may include a plurality of layers. For example, the neural network may include an input layer, a hidden layer, and an output layer.

The tympanum image processing apparatus according to an embodiment may extract the above-described tympanum outline and earwax region by applying the first machine learning model including the neural network to data corresponding to the tympanum image. For example, the tympanum image processing apparatus may input the data corresponding to the tympanum image to the input layer of the neural network. The tympanum image processing apparatus may propagate the data corresponding to the tympanum image through one or more layers from input data to the output layer. The data corresponding to the tympanum image may be extracted as abstracted feature data (e.g., a feature vector) during propagation, and the tympanum image processing apparatus may individually generate an output image indicating pixels corresponding to the tympanum outline and an output image indicating pixels corresponding to the earwax region from the feature data. However, this is only an example, and the structure of the first machine learning model is not limited to the above-described neural network.

A training apparatus may obtain the neural network from an internal database stored in a memory, or may obtain the neural network by reception thereof from an external server via a communication unit. The training apparatus may be an apparatus implemented independently from the tympanum image processing but is not limited thereto and may be integrated into the tympanum image processing apparatus.

The training apparatus according to an embodiment may train at least a portion of the neural network through supervised learning. The training apparatus may be implemented by a software module, a hardware module, or a combination thereof. The supervised learning may be a scheme of inputting, to the neural network, a training input of training data together with a training output corresponding to the training input and of updating connection weights of connection lines so that output data corresponding to the training output of the training data is output. The training data may refer to a dataset including a plurality of training pairs. For example, a training pair may include a training input and a training output, and the training output may be a value (e.g., a ground truth) to be output from the training input that forms the pair with the training output. Accordingly, the training data may include a plurality of training inputs and a training output mapped to each of the plurality of inputs.

However, training is not limited to supervised learning, and the training apparatus may train at least a portion of the neural network through unsupervised learning. The unsupervised learning may calculate loss based on an output of forward propagating the training input of the training data and may refer to a scheme of updating the connection weights of the connection lines so that the loss decreases.

The training apparatus may repeatedly change the connection weights based on the result of an objective function defined to measure how close to optimal the currently set connection weights are and may repeatedly perform training. The objective function may be, for example, a loss function for calculating loss between an output value actually output by the neural network based on the training input of the training data and an expected value desired to be output. The training apparatus may update the connection weights to reduce a value of the loss function.

For example, when the training apparatus is integrated into the tympanum image processing apparatus, the processor may repeatedly update a parameter of the first machine learning model so that an objective function value between temporary output data including the tympanum outline and the earwax region extracted using the first machine learning model from the training tympanum image and ground truth data converges.

As shown in FIG. 3, when the entire tympanum is included in the angle of view of the image, the tympanum outline 322 of the tympanum image 310 about the extracted entire tympanum may be extracted to have an elliptical shape.

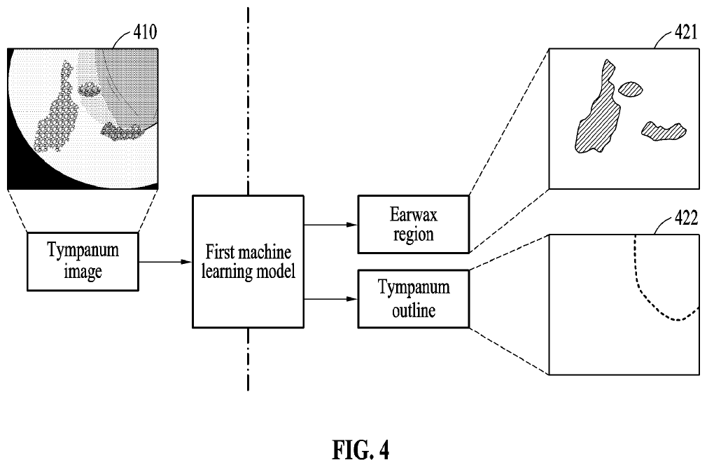

FIG. 4 illustrates a tympanum image 410 about a portion of the tympanum and a tympanum outline 422 and an earwax region 421 extracted from the tympanum image 410 using the first machine learning model, according to an embodiment. As shown in FIG. 4, when only a portion of the tympanum is included in the angle of view of the image, the tympanum outline 422 of the tympanum image 410 about a portion of the tympanum may not have an elliptical shape.

FIG. 5 illustrates an operation in which the tympanum image processing apparatus determines a target image depending on whether a tympanum image is about the entire tympanum, according to an embodiment.

As described above in operation 230 of FIG. 2, a processor according to an embodiment may obtain a target image about the entire tympanum, a tympanum outline of the target image, and an earwax region of the target image based on the tympanum outline of the tympanum image.

For example, in operation 510, the processor may determine whether the tympanum image is about the entire tympanum. According to an embodiment, the processor may determine whether the tympanum image is about the entire tympanum using an image processing technique for the tympanum image. For example, the processor may determine whether the tympanum outline of the tympanum image is an elliptical shape using the image processing technique. In response to determining that the tympanum outline of the tympanum image is an elliptical shape, the processor may determine that the tympanum image is about the entire tympanum. In response to determining that the tympanum outline of the tympanum image is not an elliptical shape, the processor may determine that the tympanum image is about a portion of the tympanum.

In operation 520, in response to determining that the tympanum image is about the entire tympanum, the processor may determine the target image based on the tympanum image. The processor may determine the tympanum image itself to be the target image but is not limited thereto. For example, the target image may be generated by pre-processing the tympanum image. The pre-processing of the tympanum image may include adjusting the size and brightness of the tympanum image. For example, the processor may generate the target image by adjusting the size of the tympanum image to a size predefined for the target image. The processor may generate the target image by adjusting the size of the tympanum region in the tympanum image to a size defined for the tympanum in the target image. The processor may perform scale adjustment (e.g., at least one of enlarging and reducing) of the tympanum image to adjust the size of the tympanum image itself and/or the size of the tympanum region. However, embodiments are not limited thereto, and the processor may adjust the size of the tympanum image by removing a portion of the tympanum image. In another example, the processor may determine, to be the target image, the tympanum image in which the brightness of the tympanum image is adjusted to the brightness of a predefined target image.

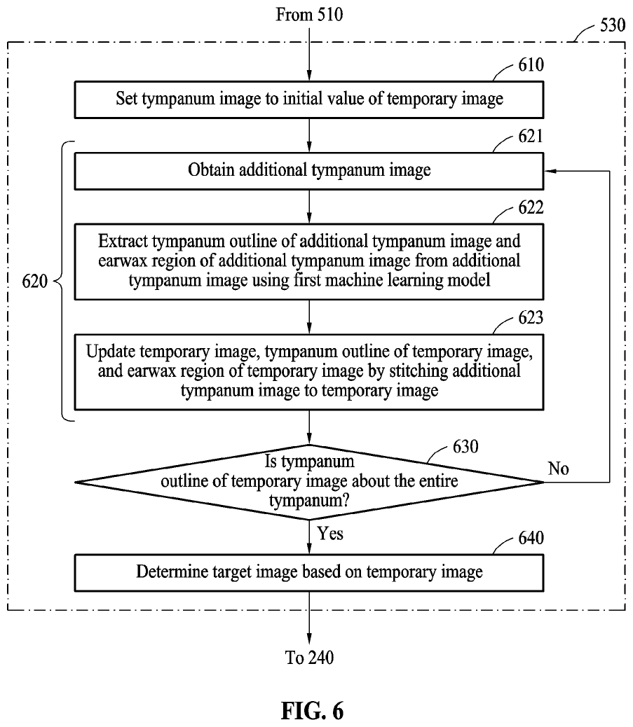

In operation 530, in response to determining that the tympanum image is about a portion of the tympanum, the processor may determine the target image based on a temporary image by generating the temporary image for the entire tympanum. Operation 530 is described in detail below with reference to FIG. 6.

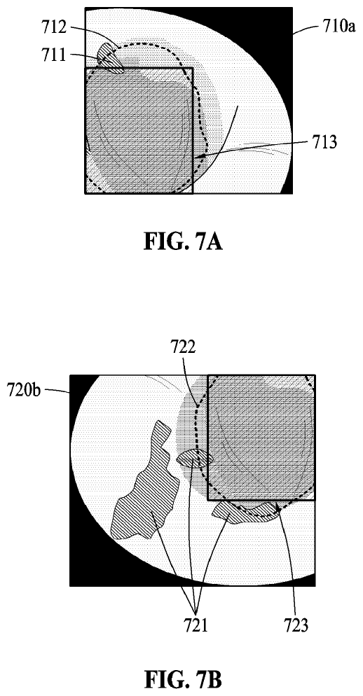

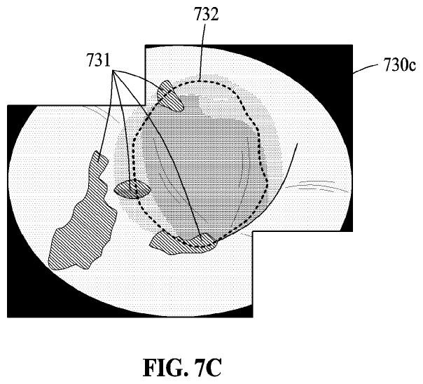

FIG. 6 illustrates a flowchart showing an operation of determining a target image based on a temporary image by generating the temporary image for the entire tympanum, according to an embodiment. FIG. 7A illustrates an earwax region 711 and a tympanum outline 712 overlaid on a tympanum image 710a for a portion of the tympanum, according to an embodiment. FIG. 7B illustrates an earwax region 721 and a tympanum outline 722 overlaid on an additional tympanum image 720b, according to an embodiment. FIG. 7C illustrates an earwax region 731 and a tympanum outline 732 overlaid on a temporary image 730c that is updated by stitching the additional tympanum image 720b of FIG. 7B to the tympanum image 710a of FIG. 7A.

In operation 610, a processor may set a tympanum image to an initial value of a temporary image.

In operation 620, the processor may stitch an additional tympanum image to the temporary image.

For example, in operation 621, the processor may obtain the additional tympanum image.

The processor according to an embodiment may request the user to capture the additional tympanum image by displaying a request guide for the additional tympanum image on a display. The processor may receive the additional tympanum image 720b from the user. However, the request guide for the additional tympanum image is not limited to the visual display on the display. For example, the request guide may include an auditory notification (e.g., voice guidance). The request guide for the additional tympanum image may include guide information indicating that the additional tympanum image needs to be captured. The request guide for the additional tympanum image may include state information indicating that an image for a portion of the tympanum is obtained. The processor may receive the additional tympanum image from the user.

An example of requesting and receiving the additional tympanum image is described but is not limited thereto. The processor according to another embodiment may receive a plurality of images. The plurality of images may be images corresponding to different frames of the image about the tympanum. The processor may determine whether the plurality of images is about the entire tympanum. In response to determining that all plurality of images is about a portion of the tympanum, the processor may select the tympanum image and the additional tympanum image from among the plurality of images.

In operation 622, the processor may extract the tympanum outline 722 of the additional tympanum image and the earwax region 721 of the additional tympanum image from the additional tympanum image using the first machine learning model.

In operation 623, the processor may update the temporary image by stitching the additional tympanum image to the temporary image. The processor according to an embodiment may identify a region that matches a portion of the tympanum image 710a in the additional tympanum image 720b. The processor may perform stitching based on regions that match each other in the tympanum image and the additional tympanum image. For example, the processor may identify a second matching region 723 of the additional tympanum image 720b that matches a first matching region 713 of the tympanum image 710a. The processor may update the temporary image 730c by stitching the additional tympanum image to the tympanum image based on the first matching region 713 and the second matching region 723. The processor may update the tympanum outline 732 of the temporary image 730c based on the tympanum outline 712 and the tympanum outline 722. For example, the processor may update the tympanum outline 732 by combining the tympanum outline 712 with the tympanum outline 722. Additionally, the processor may update the earwax region 731 of the temporary image 730c based on the earwax region 711 and the earwax region 721. For example, the processor may update the earwax region 731 by combining the earwax region 711 with the earwax region 721.

In operation 630, the processor may determine whether the temporary image is about the entire tympanum. In response to determining that the temporary image is about a portion of the tympanum, the processor may repeat operations 620 and 630. For example, the processor may repeatedly stitch the additional tympanum image to the temporary image until the temporary image is about the entire tympanum.

In operation 640, in response to determining that the temporary image is about the entire tympanum, the processor may determine the target image based on the temporary image. The processor according to an embodiment may determine the target image based on the temporary image, similar to the description of operation 520 in FIG. 5.

The processor may determine the temporary image itself to be the target image but is not limited thereto. For example, the processor may generate the target image by pre-processing the temporary image. The pre-processing of the temporary image may include adjusting the size and brightness of the temporary image. Herein, the size of the image may refer to a vertical length (e.g., a height), a horizontal length (e.g., a width), and an area occupied by the corresponding image on the display screen on which the image is output. When the image adjusts the size to be visualized on the display screen, the size to be visualized of the image may be adjusted while the ratio of the vertical length and horizontal length is maintained in the image.

For example, the processor may generate the target image by adjusting the size of the temporary image to a size predefined for the target image. The processor may generate the target image by adjusting the size of the tympanum region in the temporary image to a size defined for the tympanum region in the target image. The processor may perform scale adjustment of the temporary image to adjust the size of the temporary image itself and/or the size of the tympanum region. The scale adjustment of the temporary image may include, for example, at least one of scale reduction and scale increase. However, embodiments are not limited thereto, and the processor may adjust the size of the temporary image by removing a portion of the temporary image. In another example, the processor may determine, to be the target image, the temporary image in which the brightness of the temporary image is adjusted to the brightness of the predefined target image.

The processor may generate a transformed image in which an abnormal region of the target image is changed to a normal region.

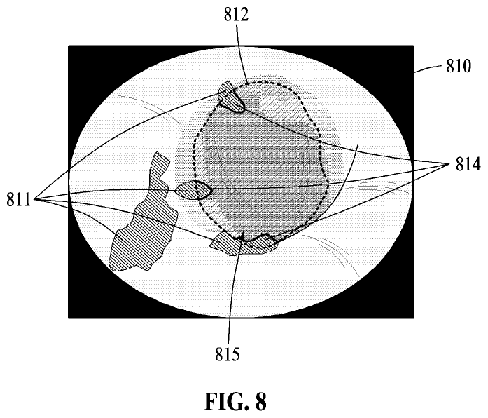

As described above in operation 240 of FIG. 2, the processor according to an embodiment may generate the transformed image in which the abnormal region of the target image is changed to the normal region. In response to a case in which a ratio of a region occluded by earwax is less than a threshold ratio compared to a region of the entire tympanum in the target image, the processor may generate the transformed image by inputting the target image to a second machine learning model. FIG. 8 illustrates a target image 810, a tympanum outline 812 of a target image, an earwax region 811 of a target image, and a region 814 where a tympanum region is occluded by an earwax region, according to an embodiment.

The target image 810 may include an image about the entire tympanum of the subject. The entire tympanum region may be a region for the entire tympanum of the subject in the target image. For example, the region of the entire tympanum in the target image may be a region 815 in the tympanum outline 812 of the target image 810. The processor may calculate the ratio of the region 814 occluded by earwax compared to the entire tympanum region in the target image. For example, the processor may calculate the above-described ratio by calculating the ratio of an area of the region 814 to an area of the region 815.

FIG. 9 illustrates a transformed image 920 generated by inputting a target image 910 about the entire tympanum to the second machine learning model and a tympanum outline 922 of a transformed image, according to an embodiment. The processor may generate a transformed image from a target image. The transformed image may be an image in which an abnormal region of the target image is changed to a normal region. The transformed image may be an image in which an earwax region of the target image is changed to the normal region.

The processor according to an embodiment may generate the transformed image 920 by inputting the target image 910 to the second machine learning model. The second machine learning model may be a model designed and trained to generate a transformed image from a target image. The machine learning model, for example, may include a neural network. The tympanum image processing apparatus according to an embodiment may generate a transformed image by applying the second machine learning model including a neural network to a target image. However, an input of the second machine learning model is not limited to the target image. For example, the processor may input the target image to the second machine learning model together with one or a combination of two or more of the tympanum outline 912 of the target image and the earwax region 911 of the target image.

For reference, when the training apparatus is integrated into the tympanum image processing apparatus, the processor may perform training of the second machine learning model. For example, the processor may calculate an objective function value between a temporary output image generated by applying the second machine learning model to a training abnormal tympanum image and a ground truth tympanum image and may repeatedly update a parameter of the second machine learning model so that the calculated objective function value converges.

The processor according to an embodiment may obtain the tympanum outline 922 of the transformed image 920 based on the tympanum outline 912 of the target image 910. The processor may calculate the tympanum outline 922 of a transformed image based on the second machine learning model. However, embodiments are not limited thereto. For example, the processor may extract a tympanum outline from a transformed image using the first machine learning model.

As described above in operation 250 of FIG. 2, a display according to an embodiment may display at least one of the transformed image and the target image. FIG. 10 illustrates a display displaying a transformed image and a target image according to an embodiment. The processor may align a tympanum region 1025 of the transformed image at a position corresponding to a position of a tympanum region 1015 of the target image on the display. The display may display the transformed image and the target image so that the tympanum region 1015 of the transformed image is aligned at a position corresponding to a position of the tympanum region 1025 of the target image. For example, the processor may align a target image 1010 and a transformed image 1020 so that a position 1030 on a first axis (e.g., the y-axis in FIG. 10) of the tympanum region 1015 of the target image 1010 and the tympanum region 1025 of the transformed image 1020 is the same on the display. The processor may align the target image 1010 and the transformed image 1020 so that positions on a second axis (e.g., the x-axis in FIG. 10) of the tympanum region 1015 of the target image 1010 and the tympanum region 1025 of the transformed image 1020 are different from each other on the display:

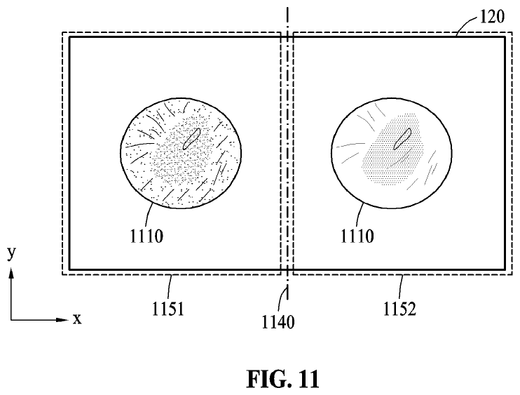

In operation 250, the display 120 may respectively display the target image and the transformed image on a first region and a second region divided by a reference line. FIG. 11 illustrates the display 120 including a first region 1151 and a second region 1152 divided by a reference line 1140, according to an embodiment. The display may visually display the reference line 1140 but is not limited thereto. The display 120 according to an embodiment may display a target image 1110 on the first region 1151 and a transformed image on the second region 1152 divided by the reference line 1140.

In FIG. 11, the reference line is illustrated as a line located at the center in the y-axis direction but is not limited thereto. For example, the reference line may be biased to one side rather than the center and may divide the first region from the second region so that the first region and the second region have different areas. In another example, the reference line may be a line in another direction (e.g., the x-axis direction in FIG. 11).

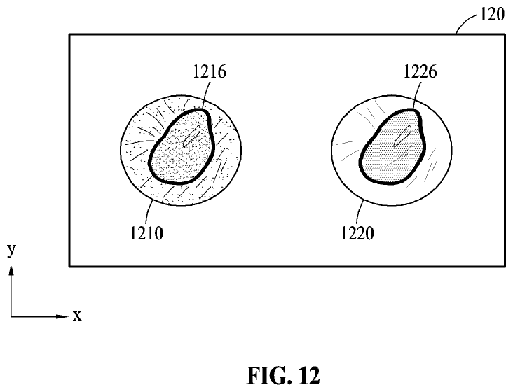

FIG. 12 illustrates a display overlaying and displaying a graphic object indicating a certain region on at least one of a target image and a transformed image, according to an embodiment.

The display 120 may overlay and display the graphic object indicating a certain region on at least one of the target image and the transformed image. The certain region may be a region to be highlighted and displayed on the display. For example, the certain region may be at least one of a tympanum outline of the target image, an earwax region of the target image, an abnormal region of the target image, a tympanum outline of the transformed image, and a region where an abnormal region of the transformed image is changed to a normal region. As shown in FIG. 12, the display 120 may overlay and display a graphic object 1216 indicating the tympanum outline of the target image on a target image 1210. The display 120 may overlay and display the graphic object 1226 indicating the tympanum outline of the transformed image on a transformed image 1220.

According to an embodiment, the processor may receive a display input from the user. For example, the processor may receive a display input associated with a certain region to be highlighted and displayed on the display. In response to the display input, the display 120 may overlay and display the graphic object indicating a certain region associated with the display input on the target image.

For example, as shown in FIG. 12, the display 120 may overlay and display the graphic object 1216 indicating the tympanum outline on the target image 1210, in response to the display input associated with the tympanum outline of the target image. The display 120 may overlay and display the graphic object 1226 on the transformed image 1220, in response to the display input associated with the tympanum outline of the transformed image.

The display 120 may include, but is not limited to, a touch display. For example, the tympanum image processing apparatus may include a housing in which physical buttons for receiving a display input from the user are disposed.

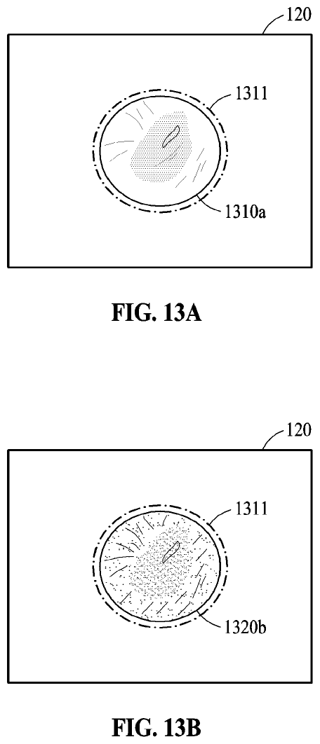

FIG. 13A illustrates a display displaying a target image 1310a in response to a target image display input, according to an embodiment. FIG. 13B illustrates the display of FIG. 13A displaying a transformed image 1320b in response to a transformed image display input.

The processor according to an embodiment may receive a display input from the user. For example, the processor may receive a display input associated with an image (e.g., a target image or a transformed image) to be displayed on the display. The display may display an image associated with the display input at a certain position 1311 in response to the display input.

For example, in FIG. 13A, the processor may receive a display input associated with the target image. The display 120 may display the target image 1310a at the position 1311 in response to the display input associated with the target image. In another example, in FIG. 13B, the processor may receive a display input associated with the transformed image. The display 120 may display the transformed image 1320b at the position 1311 in response to the display input associated with the transformed image.

The display 120 may include, but is not limited to, a touch display. For example, the tympanum image processing apparatus may include a housing in which physical buttons for receiving a display input from the user are disposed.

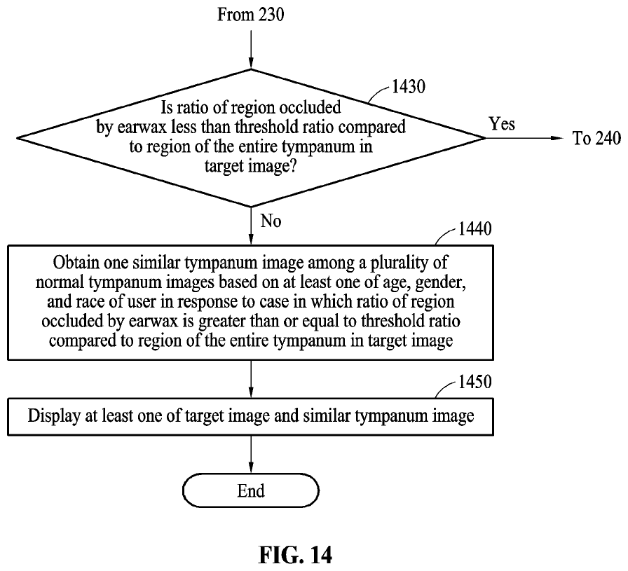

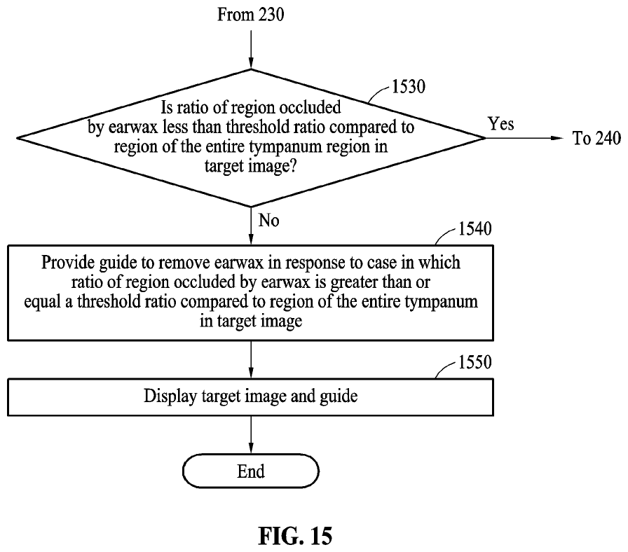

Although FIGS. 2 to 13 mainly illustrate examples in which the ratio of a region occluded by earwax is less than a threshold ratio compared to a region of the entire tympanum in the target image, examples in which the above-described ratio is greater than or equal to the threshold ratio are described below with reference to FIGS. 14 and 15.

FIG. 14 illustrates a flowchart of a tympanum image processing method that displays a target image and a similar tympanum image in response to a case in which the ratio of a region occluded by earwax is greater than or equal to a threshold ratio compared to a region of the entire tympanum in the target image, according to an embodiment.

In operation 1440, the processor may obtain one similar tympanum image among a plurality of normal tympanum images based on at least one of age, gender, and race of the subject in response to a case in which the ratio of a region occluded by earwax is greater than or equal to a threshold ratio compared to a region of the entire tympanum in the target image.

In operation 1450, the display may display at least one of the target image and the similar tympanum image.

The tympanum image processing apparatus according to an embodiment may include a memory. The plurality of normal tympanum images may be stored in the memory. Data associated with the normal tympanum image may also be stored in the memory. For example, a tympanum outline and an earwax region of the normal tympanum image corresponding to each normal tympanum image may be stored in the memory together with the normal tympanum image. In operation 1440, the processor may select one similar tympanum image from among the plurality of normal tympanum images stored in the memory.

The tympanum image processing apparatus according to an embodiment may include a communication unit. In operation 1440, the communication unit may transmit a search request for the similar tympanum image to an external server. The search request according to an embodiment may include data related to the subject. For example, data related to at least one of age, gender, and race of the subject may be included. The search request according to another embodiment may include data related to the target image. For example, data related to the target image may include at least one of the brightness of the image, the tympanum outline of the target image, and an area occupied by the tympanum region of the target image. The external server may search for one similar tympanum image among the plurality of normal tympanum images based on the search request. The communication unit may receive the similar tympanum image found from the external server. The communication unit may receive the tympanum outline and the earwax region of the found similar tympanum image together with the found similar tympanum image.

The processor may obtain one similar tympanum image among the plurality of normal tympanum images based on the similarities between the target image and each normal tympanum image. For example, the processor may receive data related to at least one of age, gender, and race of the user. The processor may calculate the similarity based on the received data. The processor may select one similar tympanum image from among the plurality of normal tympanum images based on the similarity. The processor may calculate the similarity based on metadata such as age, gender, and race of the user, but is not limited thereto. For example, the processor may calculate the similarity based on characteristics of the tympanum image itself, such as the brightness of the tympanum image, the size of the tympanum region, and the tympanum outline. It is mainly described that the processor selects the similar tympanum image. However, embodiments are not limited thereto. For example, the external server may search for one similar tympanum image based on the similarities between the target image and each normal tympanum image.

In operation 1450, similar to the description on the display of the target image and transformed image with reference to FIGS. 10 to 13, the display may display the target image and the similar tympanum image.

FIG. 15 illustrates a flowchart of a tympanum image processing method that displays a target image and a guide in response to a case in which the ratio of a region occluded by earwax is greater than or equal to a threshold ratio compared to a region of the entire tympanum in the target image, according to an embodiment.

In operation 1540, the processor may provide a guide to remove earwax in response to a case in which the ratio of a region occluded by earwax is greater than or equal to a threshold ratio compared to a region of the entire tympanum in the target image. The earwax removal guide may include guidance information that induces the user to remove earwax at a target part. For example, the earwax removal guide may include content on re-capturing the tympanum image after removing earwax and content explaining the cause of not generating the transformed image. Additionally, the earwax removal guide may include information about the ratio of a region occluded by earwax compared to a region of the entire tympanum in the target image. For example, the earwax removal guide may include content that the transformed image may not be generated because the ratio of a region occluded by earwax is greater than or equal to a threshold ratio compared to a region of the entire tympanum in the target image.

However, the earwax removal guide is not limited to the visual display on the display. For example, the earwax removal guide may include an auditory notification (e.g., voice guidance).

In operation 1550, the display may display at least one of the target image and the guide. The processor may receive a display input from the user similarly to that described above with reference to FIGS. 12 and 13. In response to the display input, the display may overlay and display the graphic object indicating a certain region on the target image. For example, the processor may receive the display input associated with the tympanum outline of the target image or the earwax region of the target image.

The embodiments described herein may be implemented using a hardware component, a software component, and/or a combination thereof. A processing device may be implemented using one or more general-purpose or special-purpose computers, such as, for example, a processor, a controller, an arithmetic logic unit (ALU), a digital signal processor (DSP), a microcomputer, a field programmable gate array (FPGA), a programmable logic unit (PLU), a microprocessor or any other device capable of responding to and executing instructions in a defined manner. The processing device may run an operating system (OS) and one or more software applications that run on the OS. The processing device also may access, store, manipulate, process, and generate data in response to execution of the software. For purpose of simplicity, the description of a processing device is used as singular: however, one skilled in the art will appreciate that a processing device may include multiple processing elements and/or multiple types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or uniformly instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software may also be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape: optical media such as CD-ROM discs, DVDs, and/or Blue-ray discs: magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter.

The above-described hardware devices may be configured to act as one or more software modules in order to perform the operations of the above-described embodiments, or vice versa.

As described above, although the embodiments have been described with reference to the limited drawings, a person skilled in the art may apply various technical modifications and variations based thereon. For example, suitable results may be achieved if the described techniques are performed in a different order and/or if components in a described system, architecture, device, or circuit are combined in a different manner and/or replaced or supplemented by other components or their equivalents.

Accordingly, other implementations are within the scope of the following claims.

1. An apparatus for processing a tympanum image, the apparatus comprising:
a processor configured to extract, from a tympanum image, a tympanum outline of the tympanum image and an earwax region of the tympanum image using a first machine learning model, obtain a target image for an entire tympanum, a tympanum outline of the target image, and an earwax region of the target image based on the tympanum outline of the tympanum image, and generate a transformed image in which an abnormal region of the target image is changed to a normal region; and
a display configured to display at least one of the transformed image and the target image so that a tympanum region of the transformed image is aligned at a position corresponding to a position of a tympanum region of the target image.

2. The apparatus of claim 1, wherein the display is configured to:
display a graphic object indicating the abnormal region on the target image; and
display a graphic object indicating a region in which the abnormal region is replaced by the normal region on the transformed image.

3. The apparatus of claim 1, wherein the processor is configured to:
determine whether the tympanum image is about an entire tympanum based on the tympanum outline of the tympanum image; and
determine the target image based on the tympanum image in response to determining that the tympanum image is about an entire tympanum.

4. The apparatus of claim 1, wherein the processor is configured to:
determine whether the tympanum image is about an entire tympanum based on the tympanum outline of the tympanum image;
obtain an additional tympanum image in response to determining that the tympanum image is about a portion of a tympanum;
extract a tympanum outline of the additional tympanum image and an earwax region of the additional tympanum image from the additional tympanum image using the first machine learning model;
update a temporary image by stitching the additional tympanum image to the tympanum image;
determine whether the temporary image is about an entire tympanum based on a tympanum outline of the temporary image; and
determine the target image based on the temporary image in response to determining that the temporary image is about an entire tympanum.

5. The apparatus of claim 1, wherein the processor is configured to generate the transformed image by inputting the target image to a second machine learning model in response to a case in which a ratio of a region occluded by earwax is less than a threshold ratio compared to a region of the entire tympanum in the target image.

6. The apparatus of claim 5, wherein the processor is configured to:
calculate an objective function value between a temporary output image generated by applying the second machine learning model to a training abnormal tympanum image and a ground truth tympanum image; and
repeatedly update a parameter of the second machine learning model so that the calculated objective function value converges.

7. The apparatus of claim 1, wherein the processor is configured to repeatedly update a parameter of the first machine learning model so that an objective function value between temporary output data comprising a tympanum outline and an earwax region extracted using the first machine learning model from a training tympanum image and ground truth data converges.

8. The apparatus of claim 1, wherein
the processor is configured to provide an earwax removal guide in response to a case in which a ratio of a region occluded by earwax is greater than or equal to a threshold ratio compared to a region of the entire tympanum in the target image, and
the display is configured to display the target image and the earwax removal guide.

9. The apparatus of claim 1, wherein
the processor is configured to select one similar tympanum image from among a plurality of normal tympanum images based on at least one of age, gender, and race of a user in response to a case in which a ratio of a region occluded by earwax is greater than or equal to a threshold ratio compared to a region of the entire tympanum in the target image, and
the display is configured to display the similar tympanum image and the target image by aligning a tympanum region of the similar tympanum image at a position corresponding to the position of the tympanum region of the target image.

10. A method of processing a tympanum image, the method comprising:
extracting, from a tympanum image, a tympanum outline of the tympanum image and an earwax region of the tympanum image using a first machine learning model;
obtaining a target image of an entire tympanum, a tympanum outline of the target image, and an earwax region of the target image based on the tympanum outline of the tympanum image;
generating a transformed image in which an abnormal region of the target image is changed to a normal region; and
displaying at least one of the transformed image and the target image so that a tympanum region of the transformed image is aligned at a position corresponding to a position of a tympanum region of the target image.

11. A computer program stored in a non-transitory computer-readable medium, the computer program being configured to perform the method of claim 10 in combination with hardware.
